(19) United States
(12) Patent Application Publication
(10) Pub. No.: US 2008/0243233 A1
(43) Pub. Date: Oct. 2, 2008

(54) Device and Methods for Treatment of Vascular Bifurcations

(75) Inventors: Shmuel Ben-Muvhar, Tirat Zvi (IL); Amir Miller, Ramat-Hasharon (IL)

Correspondence Address: DARBY & DARBY P.C., P.O. BOX 770, Church Street Station, New York, NY, 10008-0770, US

(73) Assignee: B-BALLOON LTD., Ariel (IL)

(21) Appl. No.: 11/815,652

(22) PCT Filed: Feb. 7, 2006
(86) PCT No.: PCT/IL06/00147

§ 371 Date: Aug. 6, 2007

(51) Int. Cl. A61F 2/06

(52) U.S. Cl. 623/1.35

(57) ABSTRACT

A method for treatment of a vascular bifurcation, where a second blood vessel (22) branches from a first blood vessel (24), the first and second blood vessels having characteristic first and second diameters such that the first diameter is greater than the second diameter. The method includes introducing a balloon (30) in the first blood vessel into proximity to the vascular bifurcation. The balloon includes an inner part (32) and a collar (34) proximal to the inner part. The collar is inflated so that the collar expands to an expanded diameter greater than the second diameter but less than an outer diameter of an ostial funnel (26) of the second blood vessel. The balloon is advanced into the second blood vessel so that the inflated collar lodges within the ostial funnel. While the inflated collar is lodged within the ostial funnel, the inner part of the balloon is inflated so as to treat the second blood vessel.

### CROSS-REFERENCE TO RELATED APPLICATION

This application claims the benefit of U.S. Provisional Patent Application 60/651,430, filed Feb. 8, 2005, which is assigned to the assignee of the present patent application and whose disclosure is incorporated herein by reference.

### FIELD OF THE INVENTION

The present invention relates generally to vascular catheterization, and specifically to intravascular balloons and stents.

### BACKGROUND OF THE INVENTION

Intravascular stents are used for various purposes, including opening occluded blood vessels. Typically, the stent is supplied in a narrow, contracted form, with a deflated balloon contained inside the stent. The stent and balloon are held at the distal end of a catheter. The operator inserts a guide wire into the blood vessel, and then slides the catheter over the wire to position the stent in the proper location. The balloon is then inflated, via a channel in the catheter, causing the stent to expand so as to be anchored in place and hold the vessel open. Once the stent has been expanded, the balloon is deflated and is withdrawn, along with the catheter, from the vessel.

It is sometimes necessary to insert a stent at the location of a bifurcation, where two blood vessels meet. In such cases, the stent must be inserted into the vessel that is to be expanded in such a way that the other vessel at the bifurcation is not blocked by the stent or damaged by the procedure. The operator performing the procedure must also take care not to dislodge plaques from either of the vessels at the bifurcation while performing the treatment.

PCT Patent Publication WO 2005/041810 A2, whose disclosure is incorporated herein by reference, describes apparatus for treatment of a vascular bifurcation. In some of the disclosed embodiments, a balloon comprises a narrow inner part, for insertion into a side vessel at a bifurcation, and a collar, which surrounds one end of the narrow inner part of the balloon. The collar is configured to inflate to a larger diameter than the inner part. During treatment, the narrow part of the balloon is inserted into the side vessel so that the collar is positioned at the ostium, where the side vessel joins the main vessel. Inflation of the balloon causes the inner part to expand within the side vessel, while the collar, whose inflated diameter is larger than the side vessel, remains in the main vessel. In one embodiment, the inflated collar serves as a stopper against the ostium, and thus aids the operating physician in positioning the stent properly at the ostium. In another embodiment, the balloon is used in implanting a stent in the side vessel, wherein one end of the stent protrudes from the side vessel into the main vessel and is expanded by the collar to a larger diameter than the rest of the stent in order to engage the ostium.

U.S. Pat. Nos. 5,607,444 and 5,868,777, whose disclosures are incorporated herein by reference, describe a method and apparatus for repairing a vessel at a bifurcation without obstructing blood flow through the bifurcation. An expandable ostial stent comprises a tubular body and a deformable flaring portion. Repair of a bifurcated vessel is accomplished by positioning the expandable ostial stent within a diseased portion of the bifurcation so that the flaring portion caps the ostium and the tubular body is seated within a side branch to the bifurcation, thereby completely repairing the vessel at the bifurcation without occluding blood flow.

U.S. Pat. No. 5,749,890, whose disclosure is incorporated herein by reference, describes a stent delivery assembly and method for stent placement in an ostial lesion. The stent delivery system comprises a stent delivery assembly having a distally-located deployment segment, which comprises a break segment which has an alterable configuration, as well as a stent-bearing segment. The break segment may be introduced into the patient in a first configuration. Then, when in proximity to the ostial lesion, the configuration of the break segment may be altered to assume a second, expanded, configuration which may be lodged against the wall of the parent conduit vessel, thereby localizing the ostium of the target vessel containing the lesion. One or more stents mounted, in a contracted configuration, on the deployment segment, may then be deployed by expanding the deployment segment. The configuration of the break segment may then be reversed to assume the first (unexpanded) configuration, and the entire assembly may be withdrawn from the patient.

U.S. Pat. No. 5,632,762, whose disclosure is incorporated herein by reference, describes a specially tapered balloon for sizing an implantable tubular stent that has been positioned within an ostium. In one application, a stent is positioned within the right coronary ostium, and the tapered balloon is positioned within the stent to provide a radial outward flare on the proximal end of the stent. In this manner, the proximal end of the stent is pressed back against the aortic wall surrounding the right coronary ostium, thereby minimizing any obstruction to blood flow in the aorta, and possibly reducing the risk of restenosis at the right coronary ostium.

U.S. Patent Application Publication 2005/0177221, whose disclosure is incorporated herein by reference, describes a cardiovascular ostial stent, which includes two portions having different degrees of expandability. The distal portion has a normal degree of expandability to support a vessel. The proximal portion has a higher degree of expandability so that it can be formed into a flange-like structure. A balloon is designed to deploy the stent in a single operation. The balloon includes distal and proximal portions having different diameters corresponding to the distal and proximal portions of the stent. The distal portion is of normal diameter to deploy the distal portion of the stent in the vessel. The proximal portion is of greater diameter to form the proximal portion of the stent into the flange-like structure. In one embodiment, a conventional stent is deployed through one branch (of a bifurcation, and then a second stent is deployed through a wall of the first stent and into the other branch. The flange-like structure on the a second stent secures the a second stent within the conventional stent.

### SUMMARY OF THE INVENTION

Embodiments of the present invention provide novel methods for treatment of vascular bifurcations, as well as stents, balloons, and ancillary components for use in such treatment. (The term “bifurcation” as used herein refers to the area where two blood vessels meet, and includes the ostium.) These methods permit medical practitioners to position stents with enhanced accuracy and ease.

There is therefore provided, in accordance with an embodiment of the present invention, a method for treatment of a vascular bifurcation, where a second blood vessel branches from a first blood vessel, the first and second blood vessels having characteristic first and second diameters such that the first diameter is greater than the second diameter, the method including:

introducing a balloon in the first blood vessel into proximity to the vascular bifurcation, the balloon including an inner part and a collar proximal to the inner part;

inflating the collar so that the collar expands to an expanded diameter greater than the second diameter but less than an outer diameter of an ostial funnel of the second blood vessel;

advancing the balloon into the second blood vessel so that the inflated collar lodges within the ostial funnel; and

while the inflated collar is lodged within the ostial funnel, inflating the inner part of the balloon so as to treat the second blood vessel.

In disclosed embodiments, the method includes fitting a stent over the inner part of the balloon, wherein introducing the balloon includes deploying the stent within the second blood vessel, and wherein inflating the inner part of the balloon causes the stent to expand so that the stent is implanted within the second blood vessel. In some embodiments, the stent includes proximal struts, and inflating the collar causes the proximal struts to expand to a size greater than the second diameter so as to engage the ostial funnel. Typically, the expanded struts do not extend into the first blood vessel.

There is also provided, in accordance with an embodiment of the present invention, a method for treatment of a vascular bifurcation, where a second blood vessel branches from a first blood vessel, the first and second blood vessels having characteristic first and second diameters such that the first diameter is greater than the second diameter, the method including:

fitting a stent over a balloon, which includes an inner part and a collar proximal to the inner part, the stent including proximal struts that extend over the collar;

introducing the balloon, with the stent fitted over the balloon, in the first blood vessel into proximity to the vascular bifurcation;

inflating the collar so as to cause the struts to bend outward to an expanded diameter greater than the second diameter;

advancing the inner part of the balloon, with the stent fitted over the balloon, into the second blood vessel so that the bent struts engage an ostium of the second blood vessel; and

while the bent struts engage the ostium, inflating the inner part of the balloon so as to expand and implant the stent within the second blood vessel.

In a disclosed embodiment, the method includes deflating the collar before the bent struts engage the ostium.

There is additionally provided, in accordance with an embodiment of the present invention, a method for treatment of a vascular bifurcation, where a second blood vessel branches from a first blood vessel, the first and second blood vessels having characteristic first and second diameters such that the first diameter is greater than the second diameter, the method including:

fixing a stent to a distal end of a delivery catheter having an axis, the delivery catheter including resilient struts protruding radially outward from the axis at a location proximal to the stent to an expanded diameter greater than the second diameter;

advancing the delivery catheter, with the stent fixed to the distal end thereof, into the first blood vessel through a guiding catheter, thus causing the struts to fold within the guiding catheter in a direction parallel to the axis while the delivery catheter is advanced through the guiding catheter;

introducing the distal end of the delivery catheter, with the stent fixed thereto, distally out of the guiding catheter into proximity to the vascular bifurcation, thus permitting the struts to protrude radially outward from the axis;

advancing the distal end of the delivery catheter, with the stent fitted thereto, into the second blood vessel so that the protruding struts engage an ostium of the second blood vessel; and

while the protruding struts engage the ostium, expanding and implanting the stent within the second blood vessel.

In some embodiments, fixing the stent includes fitting the stent over a balloon at the distal end of the delivery catheter, and expanding the stent includes inflating the balloon.

In a disclosed embodiment, the method includes, after implanting the stent, withdrawing the delivery catheter from the first blood vessel through the guiding catheter, thus causing the struts to fold within the guiding catheter while the delivery catheter is withdrawn.

There is further provided, in accordance with an embodiment of the present invention, a method for treatment of a vascular bifurcation, where a second blood vessel branches from a first blood vessel, the first and second blood vessels having characteristic first and second diameters such that the first diameter is greater than the second diameter, the method including:

fixing a self-expanding stent to a distal end of a catheter having an inflatable collar;

deploying the distal end of the catheter, while holding the stent in a compact state, in the first blood vessel in proximity to the vascular bifurcation;

inflating the collar while the distal end of the catheter is in proximity to the vascular bifurcation so that the collar expands to an expanded diameter greater than the second diameter;

advancing the catheter into the second blood vessel so that the inflated collar engages an ostium of the second blood vessel; and

while the inflated collar engages the ostium, releasing the stent so that the stent expands, thus implanting the stent within the second blood vessel.

In a disclosed embodiment, fixing the self-expanding stent includes fitting a sleeve over the self-expanding stent so as to hold the stent in the compact state, and releasing the stent includes withdrawing the sleeve from the second blood vessel while the stent remains within the second blood vessel. Typically, withdrawing the sleeve includes pulling the sleeve through a hub of the collar while the inflated collar engages the ostium.

In some embodiments, inflating the collar includes inflating the collar to an expanded diameter greater than the second diameter but less than an outer diameter of an ostial funnel of the second blood vessel, and advancing the catheter includes advancing the collar into the second blood vessel so that the inflated collar lodges within the ostial funnel.

There is moreover provided, in accordance with an embodiment of the present invention, a method for treatment of a vascular bifurcation between first and second blood vessels, the method including:

using a catheter, introducing first and second balloon chambers into the first and second blood vessels, respectively, in an area of the bifurcation; and

treating the bifurcation by selectively inflating the first and second balloon chambers with a fluid via the catheter using a dual-channel manifold coupled to the catheter; and

configuring the manifold so as to automatically limit a quantity of the fluid with which at least one of the first and second balloon chambers is filled to a predetermined volume.

In a disclosed embodiment, selectively inflating the first and second balloon chambers includes inflating the first balloon chamber to the predetermined volume, and thereafter inflating the second balloon chamber. The method may also include deflating the first and second balloon chambers simultaneously.

In some embodiments, the first balloon chamber is configured as a collar surrounding a part of the second balloon chamber, and introducing the first and second balloon chambers includes inserting the second balloon chamber into the second blood vessel, while the part of the balloon surrounded by the collar remains in the first blood vessel, and treating the bifurcation includes inflating the collar and bringing the inflated collar into engagement with an ostium of the second blood vessel before inflating the second balloon chamber.

There is furthermore provided, in accordance with an embodiment of the present invention, a method for producing a balloon, including:

fastening an inner part of the balloon around the catheter so as to seal the inner part to the catheter;

forming an inflatable collar around the inner part by fastening a first end of the collar around the inner part so as to seal the collar to the inner part, and fastening a second end of the collar around the catheter so as to seal the collar to the catheter.

In one embodiment, the method includes providing inflation ports in the catheter through which the inner part and the collar can be independently inflated.

Typically, fastening the first end of the collar includes folding the first end inward toward the catheter, and sealing the folded first end to a neck of the inner part of the balloon.

There is also provided, in accordance with an embodiment of the present invention, a method for treatment of a vascular bifurcation between first and second blood vessels, the method including:

fixing first and second balloon chambers to a distal end of a catheter;

fitting a retainer over the first and second balloon chambers;

advancing the distal end of the catheter, while the first and second balloon chambers are contained within the retainer, into a vicinity of the bifurcation;

releasing the first and second balloon chambers from the retainer in the vicinity of the bifurcation; and

after releasing the first and second balloon chambers, deploying and inflating the first and second balloon chambers in the first and second blood vessels, respectively, in order to treat the vascular bifurcation.

In one embodiment, the retainer includes a sleeve. In another embodiment, the retainer includes a wire, which is wound around the first and second balloon chambers. Typically, releasing the first and second balloon chambers includes shifting the retainer in a proximal direction relative to the first and second balloon chambers.

There is additionally provided, in accordance with an embodiment of the present invention, a method for treatment of a vascular bifurcation where a second blood vessel branches from a first blood vessel, the method including:

implanting in the first blood vessel a first stent having a lateral opening, such that the lateral opening is aligned with the second blood vessel;

fitting a second stent over a balloon, which includes an inner part and a collar proximal to the inner part, the stent including proximal struts that extend over the collar;

positioning the balloon, with the second stent fitted over the balloon, in the lateral opening such that the inner part of the balloon protrudes through the lateral opening into the second blood vessel;

inflating the collar within the first blood vessel so as to cause the struts to bend outward and engage the first stent in proximity to the lateral opening, whereby the second stent is positioned at a desired location in the second blood vessel; and

while the bent struts engage the first stent, inflating the inner part of the balloon so as to expand and implant the second stent within the second blood vessel.

Typically, positioning the balloon includes inserting a guide wire through the first blood vessel into the second blood vessel via the lateral opening in the first stent, and advancing the balloon over the guide wire.

In a disclosed embodiment, the proximal struts have differing respective lengths, which are chosen responsively to a branching angle of the bifurcation.

There is further provided, in accordance with an embodiment of the present invention, apparatus for treatment of a vascular bifurcation, where a second blood vessel branches from a first blood vessel, the first and second blood vessels having characteristic first and second diameters such that the first diameter is greater than the second diameter, the apparatus including:

a balloon, including an inner part and a collar proximal to the inner part, wherein the collar is coupled to be inflated to an expanded diameter greater than the second diameter but less than an outer diameter of an ostial funnel of the second blood vessel;

a catheter, which has a distal end to which the balloon is fixed, and which is configured for insertion through the first blood vessel into proximity with the vascular bifurcation and is operable to inflate the collar and to advance the balloon into the second blood vessel so that the inflated collar lodges within the ostial funnel, and to inflate the inner part of the balloon while the inflated collar is lodged within the ostial funnel so as to treat the second blood vessel.

There is moreover provided, in accordance with an embodiment of the present invention, apparatus for treatment of a vascular bifurcation, where a second blood vessel branches from a first blood vessel, the first and second blood vessels having characteristic first and second diameters such that the first diameter is greater than the second diameter, the apparatus including:

a balloon, which is fixed to the distal end of the catheter and includes an inner part and a collar proximal to the inner part;

a stent, which is fitted over the inner part of the balloon and includes proximal struts that extend over the collar; and

a catheter, which has a distal end to which the balloon is fixed, and which is configured for insertion through the first blood vessel into proximity with the vascular bifurcation and is operable to deploy the balloon, with the stent fitted over the balloon, in the first blood vessel in proximity to the vascular bifurcation, to inflate the collar so as to cause the struts to bend outward to an expanded diameter greater than the second diameter, then to advance the inner part of the balloon, with the stent fitted over the balloon, into the second blood vessel so that the bent struts engage an ostium of the second blood vessel, and to inflate the inner part of the balloon while the bent struts engage the ostium so as to expand and implant the stent within the second blood vessel.

There is furthermore provided, in accordance with an embodiment of the present invention, apparatus for treatment of a vascular bifurcation, where a second blood vessel branches from a first blood vessel, the first and second blood vessels having characteristic first and second diameters such that the first diameter is greater than the second diameter, the apparatus including:

a stent;

a guiding catheter, which is configured to be introduced into the first blood vessel in proximity to the vascular bifurcation;

a delivery catheter, which has an axis and a distal end to which the stent is fixed, and which is configured to pass through the guiding catheter into the first blood vessel, and which includes resilient struts at a location proximal to the stent,

wherein passing the delivery catheter through the guide catheter causes the struts to fold in a direction parallel to the axis, and wherein upon emergence of the distal end of the delivery catheter distally out of the guiding catheter in proximity to the vascular bifurcation, the resilient struts protrude radially outward from the axis and engage an ostium of the second blood vessel while the stent is inserted into the second blood vessel.

In a disclosed embodiment, the apparatus includes a sleeve fitted around the delivery catheter so as to hold the resilient struts parallel to the axis while passing the delivery catheter through the guide catheter.

There is also provided, in accordance with an embodiment of the present invention, apparatus for treatment of a vascular bifurcation, where a second blood vessel branches from a first blood vessel, the first and second blood vessels having characteristic first and second diameters such that the first diameter is greater than the second diameter, the apparatus including:

a self-expanding stent;

a catheter, having a distal end to which the stent is fixed in a compact state and which is configured for insertion through the first blood vessel into proximity with the vascular bifurcation so that the stent is inserted into the second blood vessel, the catheter including an inflatable collar proximal to the distal end,

wherein the catheter is operable to inflate the collar while the distal end of the catheter is in proximity to the vascular bifurcation so that the collar expands to an expanded diameter greater than the second diameter and engages an ostium of the second blood vessel while the stent is released within the second blood vessel so that the stent expands, thus implanting the stent within the second blood vessel.

There is additionally provided, in accordance with an embodiment of the present invention, apparatus for vascular treatment, the apparatus including:

first and second balloon chambers;

a catheter, having distal and proximal ends and containing first and second lumens coupled respectively to the first and second balloon chambers at the distal end of the catheter; and

a manifold including first and second fluid channels coupled respectively to the first and second lumens at the proximal end of the catheter and operative to selectively inflate the first and second balloon chambers with a fluid via the catheter while automatically limiting a quantity of the fluid with which at least one of the first and second balloon chambers is filled to a predetermined volume.

There is further provided, in accordance with an embodiment of the present invention, a medical device, including:

a catheter having a distal end; and

a dual-chamber balloon fixed to the distal end of the catheter, the balloon including:

an inner part, which is fastened around the distal end of the catheter so as to seal the inner part to the catheter; and

an inflatable collar, which is formed around the inner part by fastening a first end of the collar around the inner part so as to seal the collar to the inner part, and fastening a second end of the collar around the catheter so as to seal the collar to the catheter.

There is moreover provided, in accordance with an embodiment of the present invention, apparatus for treatment of a vascular bifurcation between first and second blood vessels, the apparatus including:

a catheter having a distal end;

first and second balloon chambers, which are fixed to the distal end of a catheter;

a retainer, which fits over and contains the first and second balloon chambers while the distal end of the catheter is advanced into a vicinity of the bifurcation, and which is operable to release the first and second balloon chambers in the vicinity of the bifurcation, so that the first and second balloon chambers can be deployed and inflated in the first and second blood vessels, respectively, in order to treat the vascular bifurcation.

There is furthermore provided, in accordance with an embodiment of the present invention, apparatus for treatment of a vascular bifurcation where a second blood vessel branches from a first blood vessel, the apparatus including:

a first stent, which has a lateral opening and is configured to be implanted in the first blood vessel such that the lateral opening is aligned with the second blood vessel;

a balloon, which includes an inner part and a collar proximal to the inner part;

a second stent, which includes proximal struts and is fitted over the balloon so that the struts extend over the collar; and

a catheter, having a distal end to which the balloon is fixed, and which is operative to position the balloon, with the second stent fitted over the balloon, in the lateral opening such that the inner part of the balloon protrudes through the lateral opening into the second blood vessel, and to inflate the collar within the first blood vessel so as to cause the struts to bend outward and engage the first stent in proximity to the lateral opening, and to inflate the inner part of the balloon while the bent struts engage the first stent so as to expand and implant the second stent within the second blood vessel.

There is also provided, in accordance with an embodiment of the present invention, a method using first and second stents for treatment of a vascular bifurcation where a first blood vessel branches from a second blood vessel, the first stent having a lateral opening, the method including:

fitting the second stent over a balloon, which includes an inner part and a collar proximal to the inner part, the stent including proximal struts that extend over the collar;

positioning the balloon, with the second stent fitted over the balloon, in the vascular bifurcation so that the inner part of the balloon protrudes into the second blood vessel while the collar is in the first blood vessel;

after positioning the balloon, inflating the collar within the first blood vessel so as to cause the struts to bend outward and engage a wall of the first blood vessel in proximity to the bifurcation, whereby the second stent is positioned at a desired location in the second blood vessel; and

while the bent struts engage the wall of the first blood vessel, inflating the inner part of the balloon so as to expand and implant the second stent within the second blood vessel; and

after implanting the second stent within the second blood vessel, implanting the first stent in the first blood vessel, such that the lateral opening is aligned with the second blood vessel and engages the bent struts.

There is additionally provided, in accordance with an embodiment of the present invention, apparatus for treatment of a vascular bifurcation where a second blood vessel branches from a first blood vessel, the apparatus including:

a first stent, which has a lateral opening and is configured to be implanted in the first blood vessel such that the lateral opening is aligned with the second blood vessel;

a balloon, which includes an inner part and a collar proximal to the inner part;

a second stent, which includes proximal struts and is fitted over the balloon so that the struts extend over the collar; and

a catheter, having a distal end to which the balloon is fixed, and which is operative to position the balloon, with the second stent fitted over the balloon, in the vascular bifurcation so that the inner part of the balloon protrudes into the second blood vessel while the collar is in the first blood vessel, and to inflate the collar within the first blood vessel so as to cause the struts to bend outward and engage a wall of the first blood vessel in proximity to the bifurcation, whereby the second stent is positioned at a desired location in the second blood vessel, and to inflate the inner part of the balloon while the bent struts engage the wall of the first blood vessel so as to expand and implant the second stent within the second blood vessel,

wherein the first stent is implanted in the first blood vessel after implanting the second stent within the second blood vessel, such that the lateral opening is aligned with the second blood vessel and engages the bent struts.

The present invention will be more fully understood from the following detailed description of the embodiments thereof, taken together with the drawings in which:

### BRIEF DESCRIPTION OF THE DRAWINGS

FIGS. 1, 2, 3A, 4 and 5 are schematic, pictorial illustrations showing stages in a procedure for implanting a stent in a vascular bifurcation, in accordance with an embodiment of the present invention;

FIG. 3B is a schematic side view of a balloon and stent within a vascular bifurcation during a stage in a procedure for implanting the stent in the bifurcation, in accordance with an embodiment of the present invention;

FIG. 6 is a schematic side view of a balloon assembly mounted on a catheter, in accordance with an embodiment of the present invention;

FIG. 7 is a schematic detail view of the assembly of FIG. 6;

FIG. 8 is a schematic, exploded view of a manifold for controlling inflation and deflation of a balloon assembly, in accordance with an embodiment of the present invention;

FIG. 9 is a schematic, pictorial illustration showing details of a flow control mechanism, in accordance with an embodiment of the present invention;

FIGS. 10-15 are schematic, pictorial illustrations showing stages in a procedure for implanting a stent in a vascular bifurcation, in accordance with another embodiment of the present invention;

FIGS. 16-19 are schematic, pictorial illustrations showing stages in a procedure for implanting a stent in a vascular bifurcation, in accordance with yet another embodiment of the present invention;

FIGS. 20-22 are schematic, pictorial illustrations showing stages in deployment of a bifurcated balloon within a vascular bifurcation, in accordance with an embodiment of the present invention;

FIG. 23 is a schematic side view of a stent for implantation in a vascular bifurcation, in accordance with an embodiment of the present invention; and

FIGS. 24-27 are schematic, pictorial illustrations showing stages in a procedure for implanting a stent in a vascular bifurcation, in accordance with still another embodiment of the present invention.

### DETAILED DESCRIPTION OF EMBODIMENTS

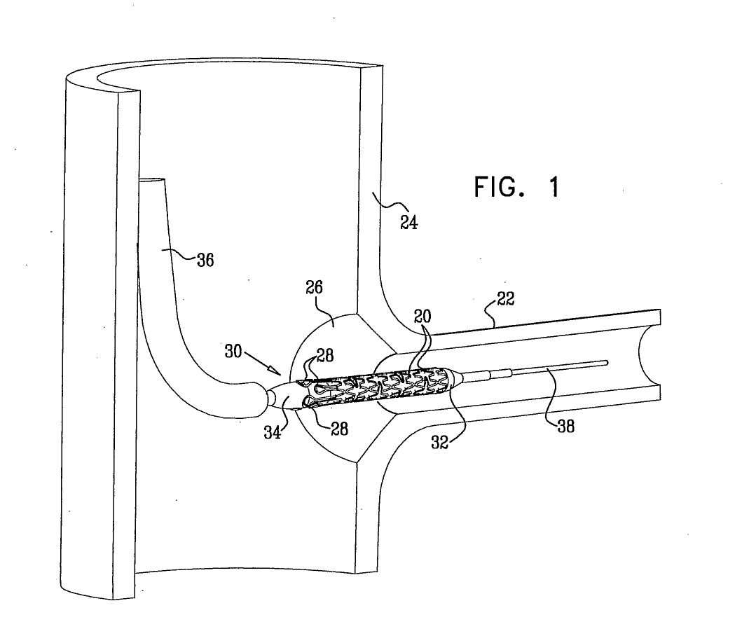

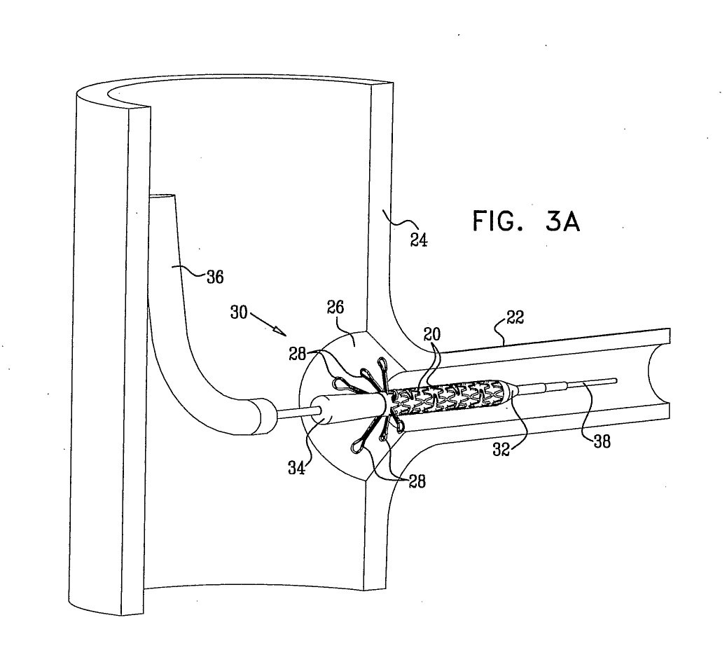

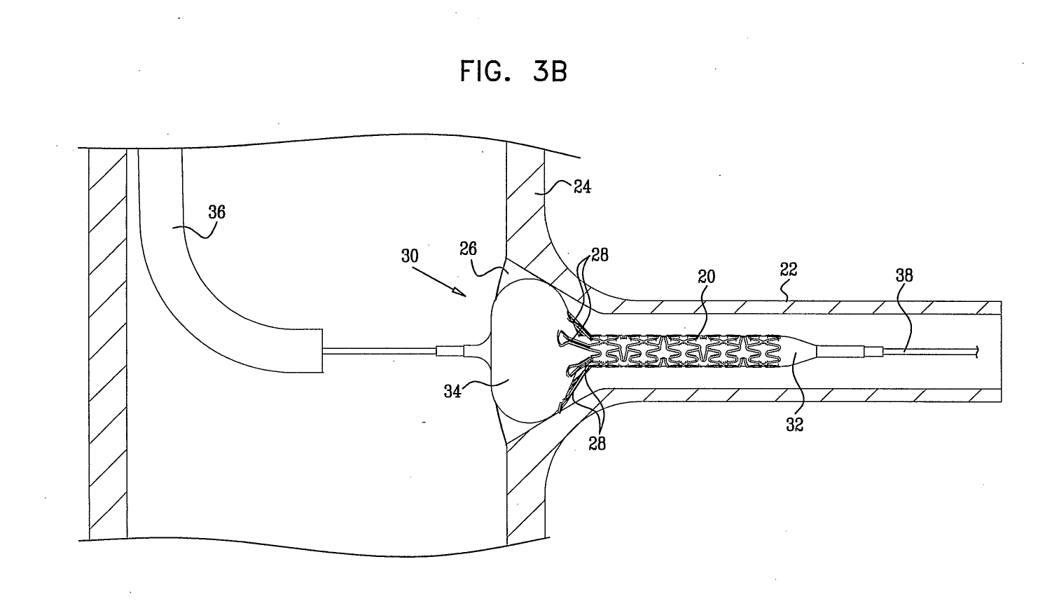

Reference is now made to FIGS. 1, 2, 3A, 3B, 4 and 5, which schematically illustrate successive stages in implantation of a stent 20 within a side vessel 22 at a bifurcation from a main vessel 24, in accordance with an embodiment of the present invention. FIGS. 1, 2, 3A, 3B, 4 and 5 are pictorial illustrations, while FIG. 3B is a side view. Stent 20 comprises struts 28 for engaging an ostium 26 of side vessel 22, as shown in these figures and described hereinbelow. This implantation procedure may be used in substantially any bifurcation, but it is especially useful for treating bifurcations from large arteries, such as the bifurcation of the coronary arteries from the ascending aorta. The inventors have found that the side vessels in such bifurcations tend to have an ostium 26 whose shape is approximately conical. Methods that have been known in the art for implanting stents in these bifurcations have failed to take this characteristic of the vascular anatomy into consideration. The present embodiment, on the other hand, is designed to accommodate the conical shape of the ostium and thus achieves more accurate and secure stenting in the area of the bifurcation than do methods and devices known in the art.

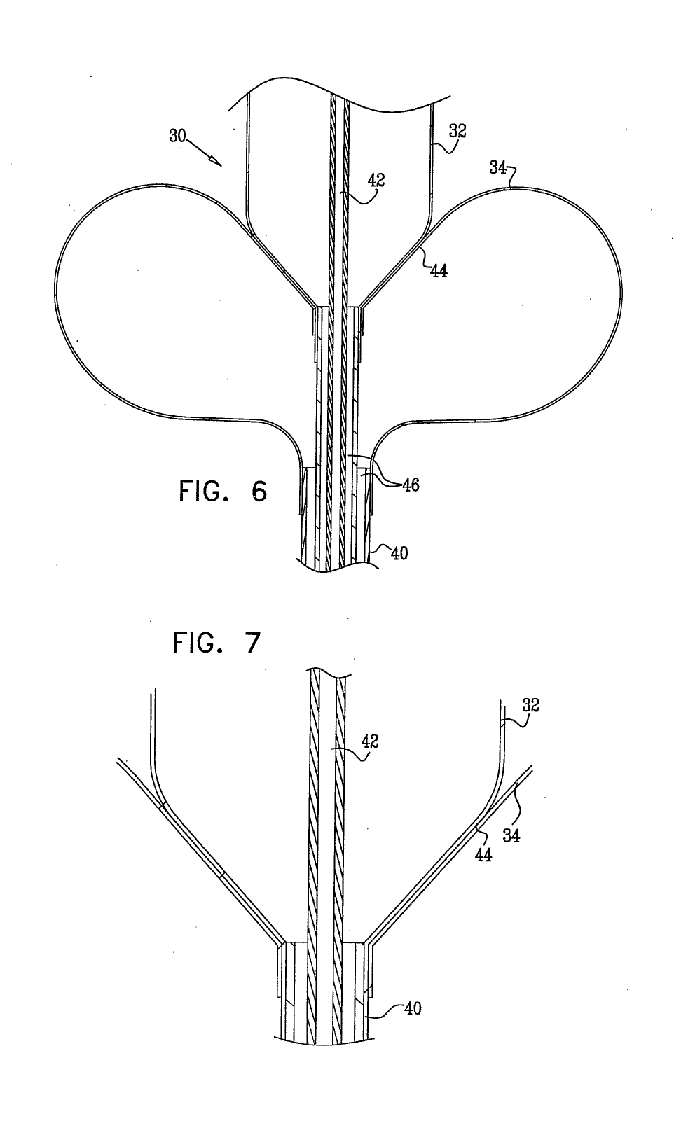

As shown in FIG. 1, stent 20, in a contracted state, is fitted and crimped over a deflated balloon 30. This balloon comprises two parts with different inflation characteristics: an inner part 32, made of semi-compliant material, and a collar 34, made of more highly compliant material, which surrounds the proximal end of inner part 32. Typically, balloon 30 comprises a biocompatible nylon-based copolymer or other suitable biocompatible material. In this embodiment, inner part 32 and collar 34 may be fabricated as separate balloons, with the collar having the general form of a toroid fitted around the inner part. Details of the construction of balloon 30 are shown in FIGS. 6 and 7 and are described hereinbelow with reference to these figures. Alternative methods that may be applied in the construction of balloon 30 are described in the above-mentioned PCT Patent Publication WO 2005/041810 A2.

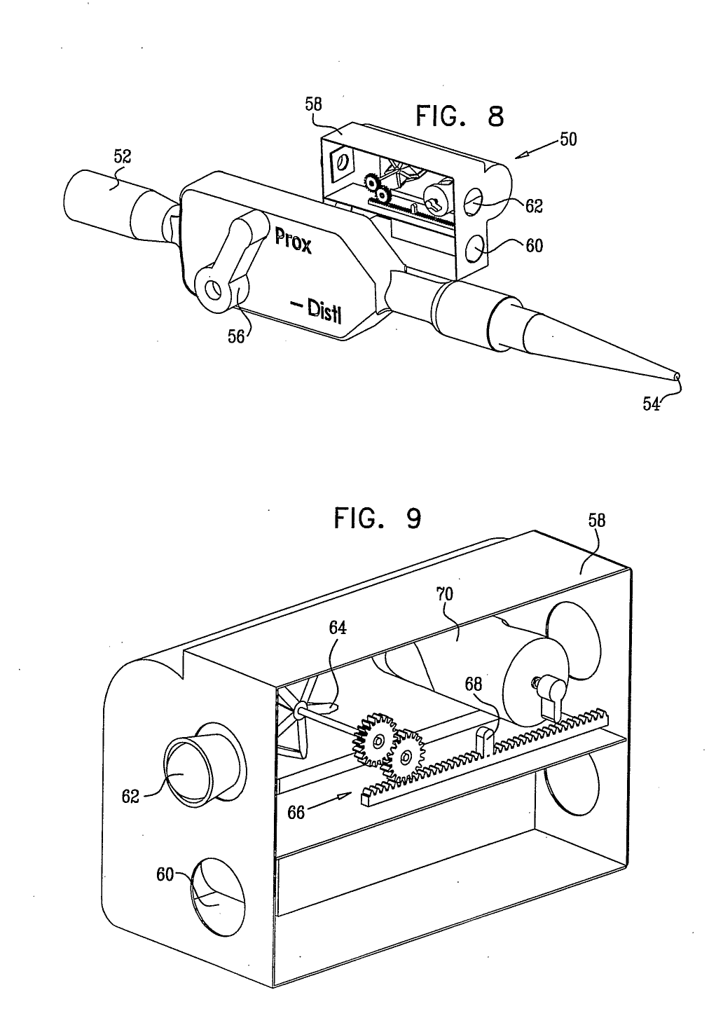

Inner part 32 and collar 34 may share a common inflation port, or they may alternatively have separate inflation ports, enabling the two parts to be inflated to different pressures. A dual-port manifold that may be used to inflate and deflate balloon 30 is described hereinbelow with reference to FIGS. 8 and 9.

Stent 20 is crimped over balloon 30 so that struts 28 extend over inner part 32 of the balloon and over at least the distal part of collar 34. The stent is delivered via a guiding catheter 36 over a guide wire 38, which has been threaded from main vessel 24 into side vessel 22. In this embodiment, balloon 30 has a central lumen with a distal opening for accommodating the guide wire. Alternatively, the catheter (in this embodiment and in the other embodiments described hereinbelow) may be adapted to operate without the use of a guide wire. In such a case, the balloon may include a built-in wire as an integral part of its assembly.

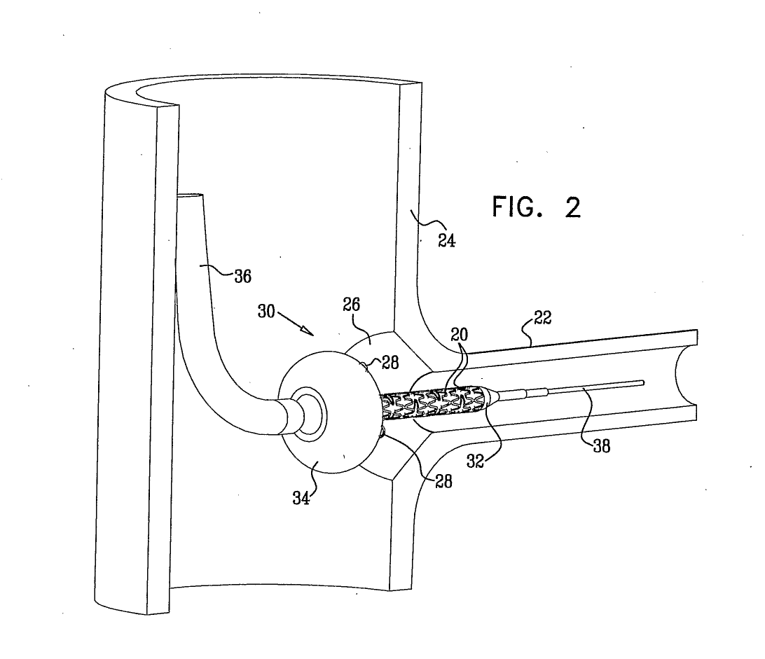

As shown in FIG. 2, once stent 20 has been introduced from catheter 36 into or near side vessel 22, collar 34 of balloon 30 is inflated, causing struts 28 to bend apart so as to reach an overall diameter greater than the diameter of side vessel 22. Two alternative options may then be used for positioning the stent in the side vessel:
As shown in FIG. 3A, collar 34 may be at least partially deflated, while struts 28 remain bent outward. The operating practitioner at this stage pushes the catheter forward, in the distal direction, so that struts 28 engage ostium 26.
As shown in FIG. 3B, the catheter may be advanced while collar 34 remains inflated, until the collar and struts 28 engage ostium 26. The collar is sized and shaped so that it fits into the conical funnel formed by ostium 26, rather than pressing against the wall of main vessel 24 as in the publications mentioned in the Background of the Invention. In other words, as can be seen in the figure, the diameter of the balloon is less than the outer diameter of the cone of the ostial funnel (also referred to hereinafter simply as the “ostial cone”), so that the balloon lodges within the cone. The collar may also be made sufficiently flexible (by controlling inflation pressure, for example) so that it deforms to fit the shape of the funnel when pushed against it. The inventors have found that choosing the proper size, shape and degree of flexibility of collar 34 are useful in ensuring accurate placement of the stent in vessel 24.

In another embodiment (not shown in the figures), the stent may simply be fitted over inner part 32 of balloon 30, without struts extending over collar 34, so that the collar alone engages the ostial cone. In any case, the use of struts 28 and/or collar 34 to engage the ostial cone ensures that stent 20 is properly positioned for expansion inside side vessel 22. The struts bend smoothly against the conical sides of the ostium, rather than protruding into main vessel 24 and bending back against the wall of the main vessel as in methods that are known in the art. Engaging the funnel, rather than the wall of the main vessel, gives the operator more precise control over the positioning of the stent, with better assurance that the stent will be properly positioned and lodged firmly in place at the end of the procedure. As in the preceding embodiment, the size, shape and flexibility of the collar are chosen to fit the size and shape of the ostial funnel.

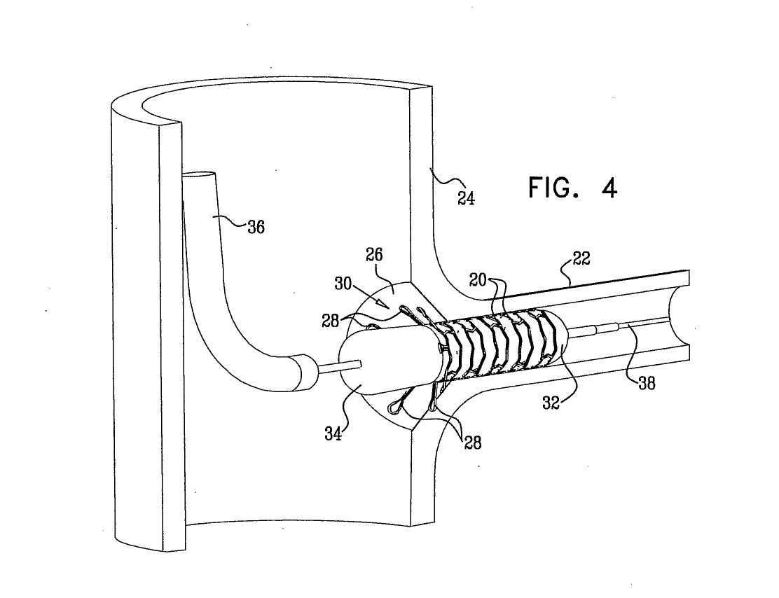

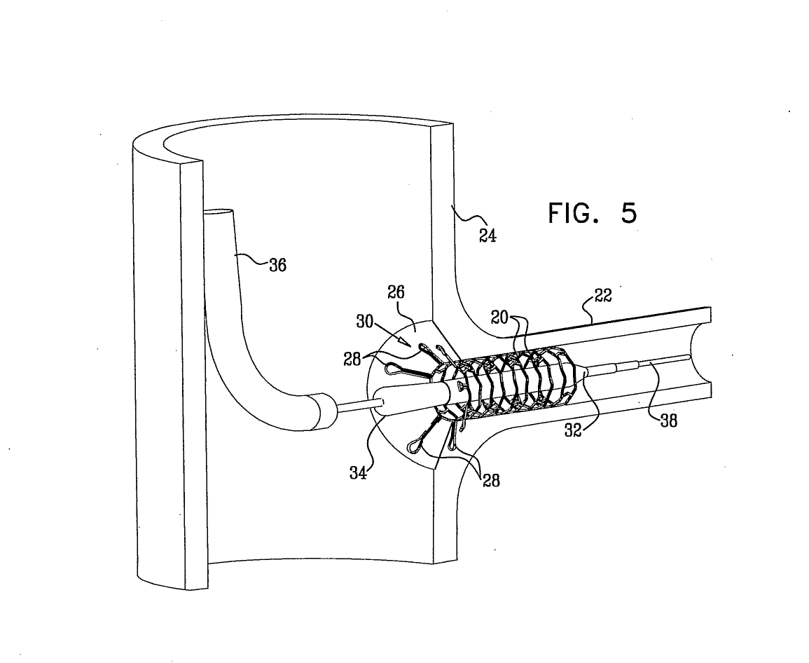

After positioning the expanded struts and/or collar against the ostium, inner part 32 of balloon 30 is fully inflated, causing the balloon to assume the shape shown in FIG. 4. Collar 34 may remain inflated during this stage, or it may alternatively be partly or completely deflated as shown in the figure. The inner part of the balloon expands the main body of stent 20, to support vessel 22. Meanwhile, struts 28 press against ostium 26 to support the ostium and help to hold the stent in place. Both the main body of stent 20 and struts 28 may elute an anti-restenosis drug into the adjacent tissue. Balloon 30 is then deflated, as shown in FIG. 5, and withdrawn from the body through vessel 24 by using catheter 36.

Reference is now made to FIGS. 6 and 7, which schematically illustrate the construction of balloon 30, in accordance with an embodiment of the present invention. FIG. 6 is a schematic side view showing the mounting of inner part 32 and collar 34 on a shaft 40 of a delivery catheter (which is located inside guiding catheter 36 in FIGS. 1-5). Shaft 40 contains a guide lumen 42, which passes through balloon 30 and is designed to be threaded over guide wire 38. The catheter also contains inflation lumens 46 with ports for selectively inflating and deflating inner part 32 and collar 34.

FIG. 7 shows details of the area in which balloon 30 is fastened to catheter 40. Typically, inner part 32 and collar 34 are fabricated from a nylon-based copolymer or other suitable biocompatible material. Inner part 32 is sealed around the circumference of catheter 40, typically by heat fusing, using a laser as a heat source, or alternatively using conductive or convective heating. Further alternatively, the seal may be produced using polymer-to-polymer glue. Collar 34 is similarly fitted around catheter 40 and is folded inward around the neck of inner part 32. The collar is then fastened and sealed to the inner part of the balloon by heat fusing or glue to form a joint 44. After forming joint 44, the proximal neck of balloon 34 is fastened (by heat fusing or glue) to catheter 40, as shown in FIG. 6. The inventors have found that this arrangement permits the two parts of balloon 30 to be formed efficiently and compactly, and gives reliable performance when the parts of the balloon are inflated.

Reference is now made to FIGS. 8 and 9, which are schematic, pictorial illustrations of a manifold 50 that may be used to inflate and deflate balloon 30, as well as other dual-chamber balloons, in accordance with an embodiment of the present invention. FIG. 8 is an exploded view of the manifold, while FIG. 9 shows details of its internal construction. At its proximal side, manifold 50 connects to a fluid supply inlet 52, which provides a pressurized supply of a suitable fluid, which may be a liquid, such as saline solution, or a suitable gas. At its distal side, the manifold connects to a dual-lumen fluid delivery outlet 54, which connects to the dual chambers of balloon 30 through catheter 36, for example.

Manifold 50 comprises two fluid channels 60 and 62, for feeding collar 34 and inner part 32 of the balloon, respectively. Each channel is controlled independently to convey a different volume of fluid, which may be determined according to the desired volume and pressure to which the corresponding part of the balloon is to be inflated. Manifold may be arranged so that both parts of the balloon are inflated and deflated simultaneously, in a single inflation or deflation procedure. In the embodiment shown in the figures, however, manifold 50 comprises a switch 56, which operates a valve (not shown) that allows the user to selectively inflate either the proximal (collar) or distal (inner part) of the balloon. Although switch 56 is shown in the figure as having the form of a lever, it may alternatively have a push-button design. Additionally or alternatively, while the valve operated by switch 56 is designed to permit the parts of the balloon to be inflated individually, one at a time, it may allow both parts of the balloon to be deflated simultaneously to facilitate rapid removal of catheter 36.

A flow control mechanism 58 applies automatic volume restriction to one of the channels—in this case channel 62, which feeds collar 34. Volume restriction is useful in ensuring that the collar is inflated to the optimal size and pressure for engaging ostium 26. (Similar volume restriction could be applied to distal channel 60, but in this embodiment the practitioner operating catheter 36 is permitted to inflate the inner part of the balloon freely to whatever pressure is desired.) The volume of fluid passing through channel 62 is measured by a turbine flow meter 64, which is connected through a rotary-to-linear transmission 66 to a rotary-actuated valve 70. After a predetermined number of turbine rotations (which represents a predefined flow volume), an actuator notch 68 driven by transmission 68 turns valve 70, thereby shutting off the fluid flow. Mechanism 58 is advantageous in terms of its simplicity and low cost. Alternatively, other types of flow control mechanisms known in the art may be used to measure and restrict the volume of fluid passing through channel 62 and/or channel 60, including electronic devices, as well as purely mechanical devices such as that shown here.

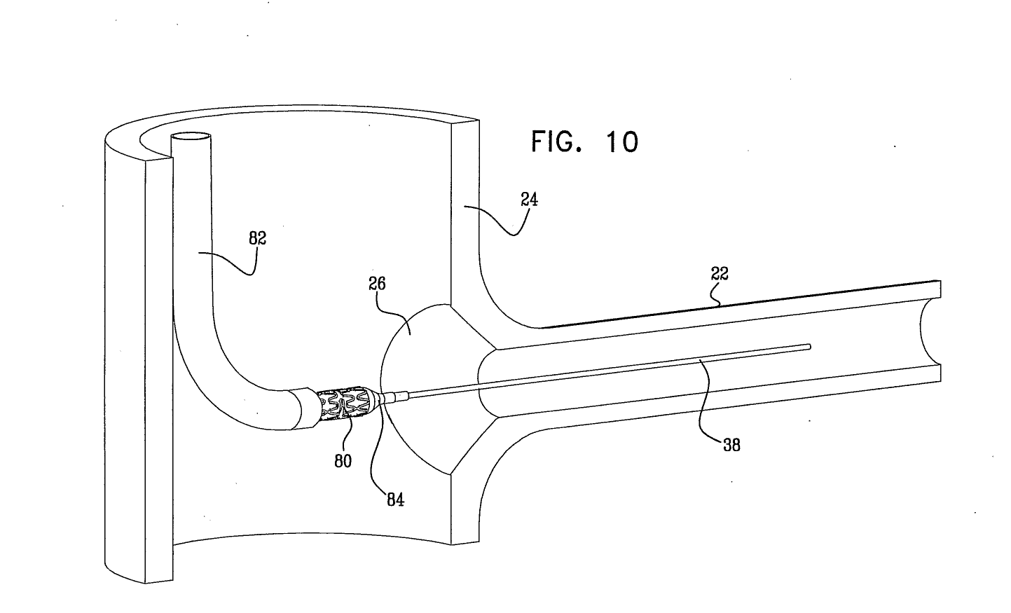

Reference is now made to FIGS. 10-15, which are schematic, pictorial illustrations showing successive stages in implantation of a stent 80 within side vessel, 22, in accordance with another embodiment of the present invention. As shown in FIG. 10, stent 80 is initially crimped over a balloon 84 and is delivered to the region of the bifurcation of vessel 22 from vessel 24 by a guiding catheter 82 along guide wire 38. When the guiding catheter reaches the vicinity of the bifurcation, the operator advances a catheter 88 (which may be referred to for clarity as the “delivery catheter”) in order to push stent 80 and balloon 84 distally out of the guiding catheter and into vessel 22, as shown in FIG. 11.

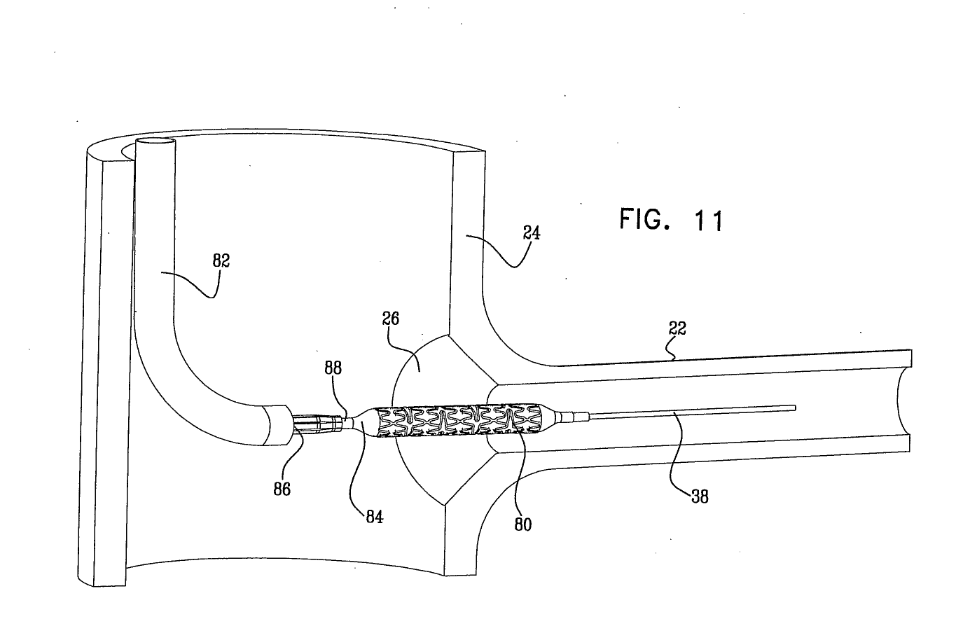

Flexible struts 86 are attached to the shaft of catheter 88 at a point proximal to balloon 84. These struts are typically made of a resilient material, such as NiTi. The struts are formed so that when released from guiding catheter 82, they spread apart, away from the axis of catheter 88, as shown in FIG. 12. As the delivery catheter is advanced, the struts are thus exposed (FIG. 11) and then released (FIG. 12), so that they protrude radially to a diameter that is greater than the diameter of side vessel 22. Prior to deployment, struts 86 may also be secured with a sleeve or sheath (not shown) in order to protect the struts from unwanted deformation. In this case, the flexible struts may be bent in the distal direction prior to deployment, rather than in the proximal direction as shown in the figures.

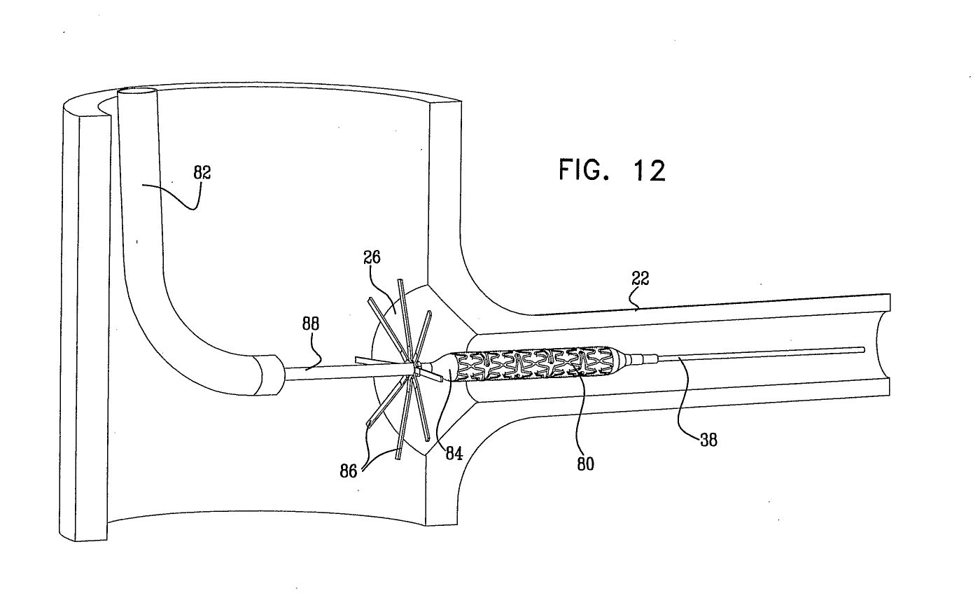

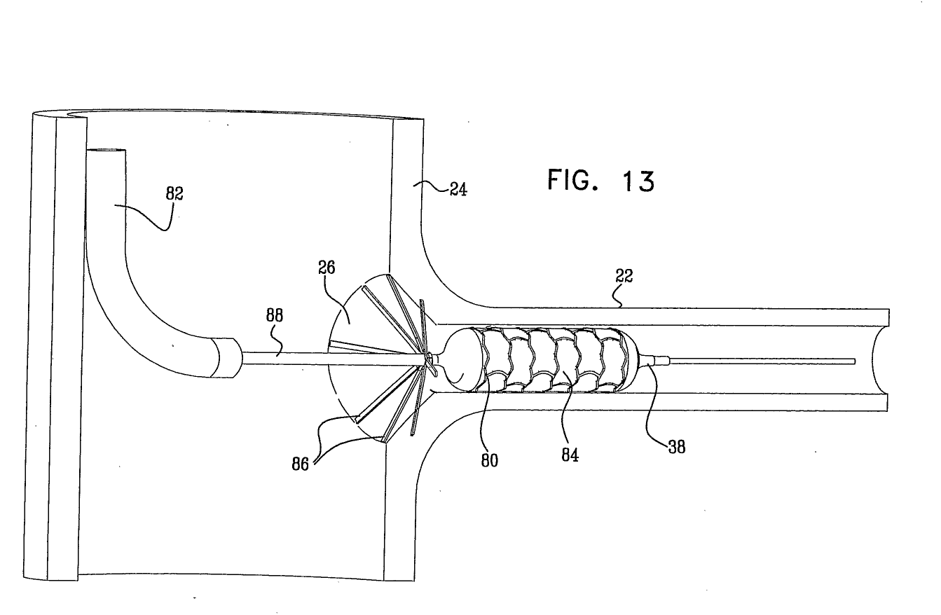

The operator continues to advance delivery catheter 88 until struts 86 engage the wall of main vessel 24 and/or ostium 26. Typically, the conical shape of the ostium, as noted above, bends the struts backward toward guidance catheter 82, as shown in FIG. 13. Struts 86 are made sufficiently stiff so that the operator can feel the resistance of the struts while pushing catheter 88 distally into side vessel 22, and can thus position stent 84 precisely at the end of the vessel near the bifurcation. Upon finding the desired position, the operator inflates balloon 84, thus expanding stent 80, as shown in FIG. 13.

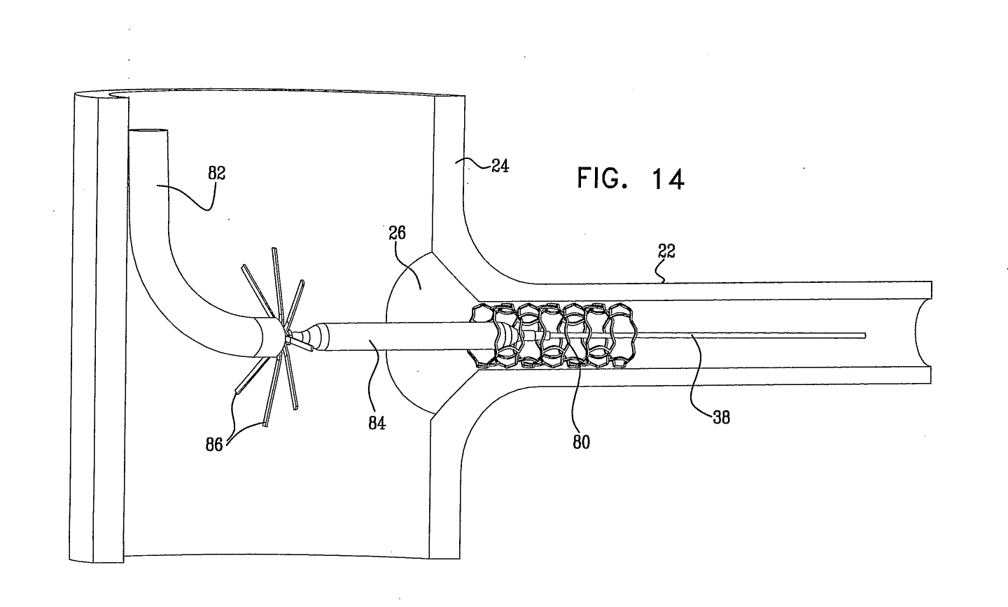

Alternatively, catheter 88 with struts 86 may be used, mutatis mutandis, in deploying a self-expanding stent, such as that shown below in FIGS. 16-19.

Once stent 80 has been fixed firmly in place in this manner, the operator deflates balloon 84 and begins to withdraw delivery catheter 88 in the proximal direction back into guiding catheter 82, as shown in FIG. 14. The neck of the guiding catheter presses against struts 86, and thus causes the struts to fold downward against deflated balloon 84, parallel to the axis of the catheter, as shown in FIG. 15. The balloon and struts are thus drawn completely into the guiding catheter, so that the entire assembly can be withdrawn easily and safely from the patient's body.

FIGS. 16-19 are schematic, pictorial illustrations showing successive stages in operation of a stent delivery system 90, in accordance with yet another embodiment of the present invention. In contrast to the preceding embodiments, in which balloon expansion was required to implant the stent, system 90 is designed to deliver a stent 94 that is self-expanding. For example, stent 94 may comprise a superelastic material, such as NiTi, as is known in the art. The stent is contained within a sleeve 96 as it is advanced through the vascular system to a vascular bifurcation by a delivery catheter 92. It is thus held in a compact state until it reaches the proper location.

The operator advances catheter 92 to position system 90 in proximity to the target bifurcation (as was shown in the preceding embodiments). For this purpose, the operator may use a radio-opaque marker under angiography, for example, in order to visualize the location of the stent. When system 90 is properly located, the operator inflates a positioning balloon 98 through a dedicated lumen (not shown) in the delivery system, so that the balloon assumes the shape shown in FIG. 17. The operator then continues to advance the catheter until balloon 98 engages the ostium, at which point the system cannot be advanced any further.

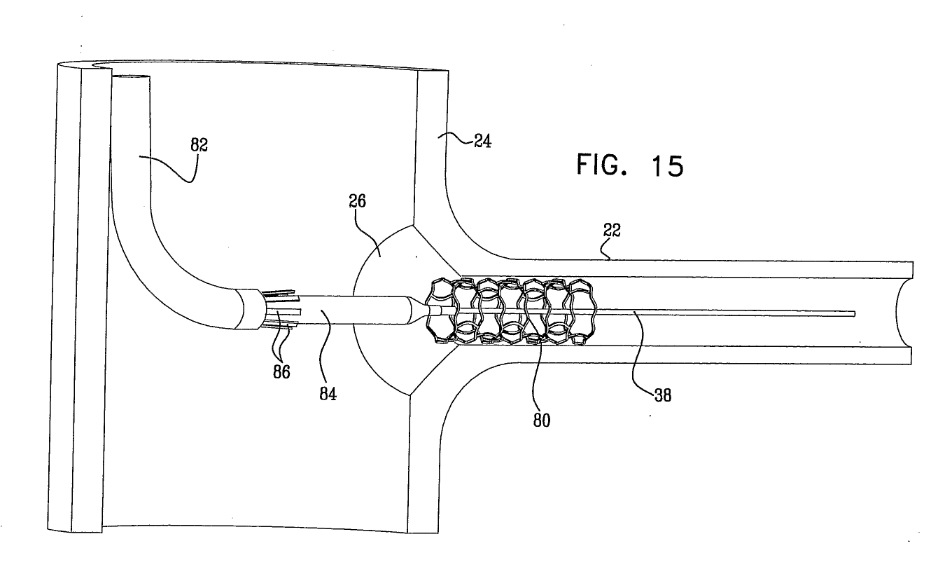

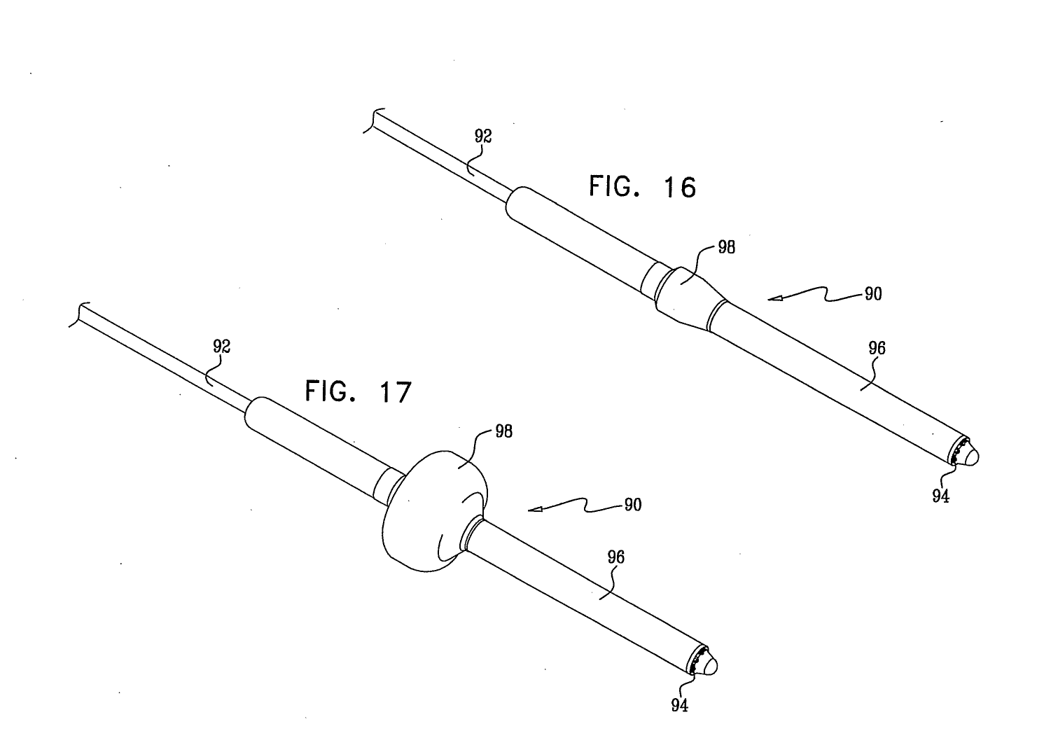

To release and deploy stent 94 in the side vessel, the operator pulls sleeve 96 back through the hub of balloon 98, as shown in FIG. 18. After the sleeve has been retracted, stent 94 expands to the full diameter of the side vessel, as shown in FIG. 19. Balloon 98 is then deflated, and system 90 is withdrawn through the vascular system while leaving stent 94 in place. Although stent 94 is shown in this figure as having a uniform expanded diameter over its entire length, the stent may alternatively have proximal struts or other elements that expand to a greater diameter in order to engage the ostial cone, as shown in the preceding embodiments.

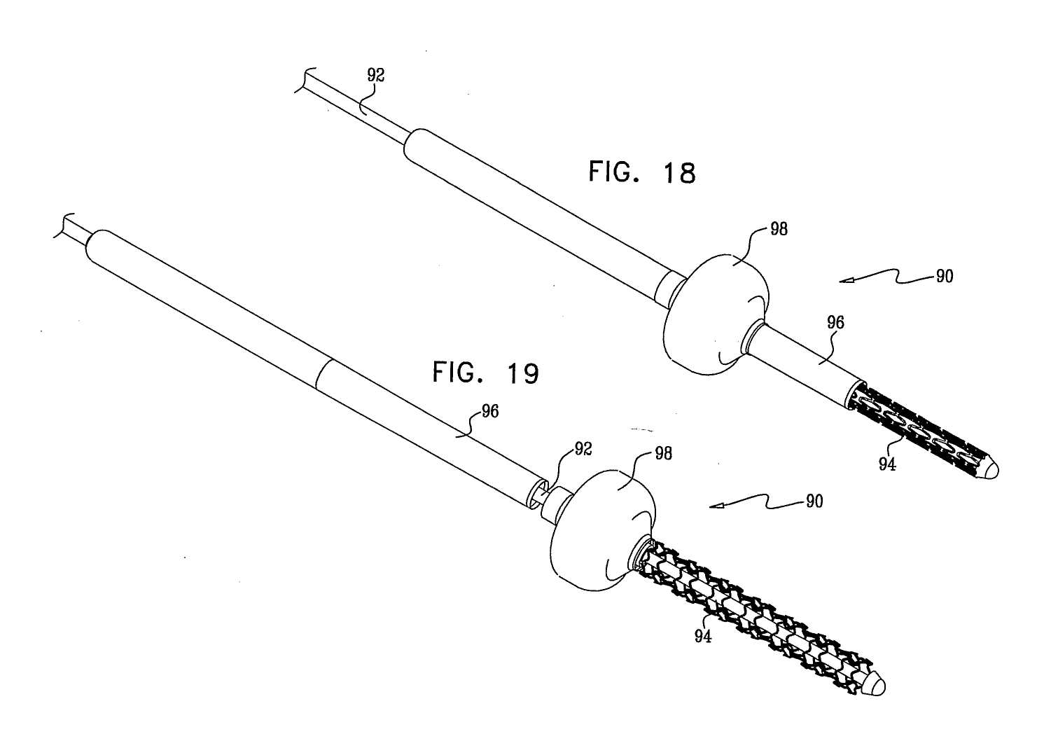

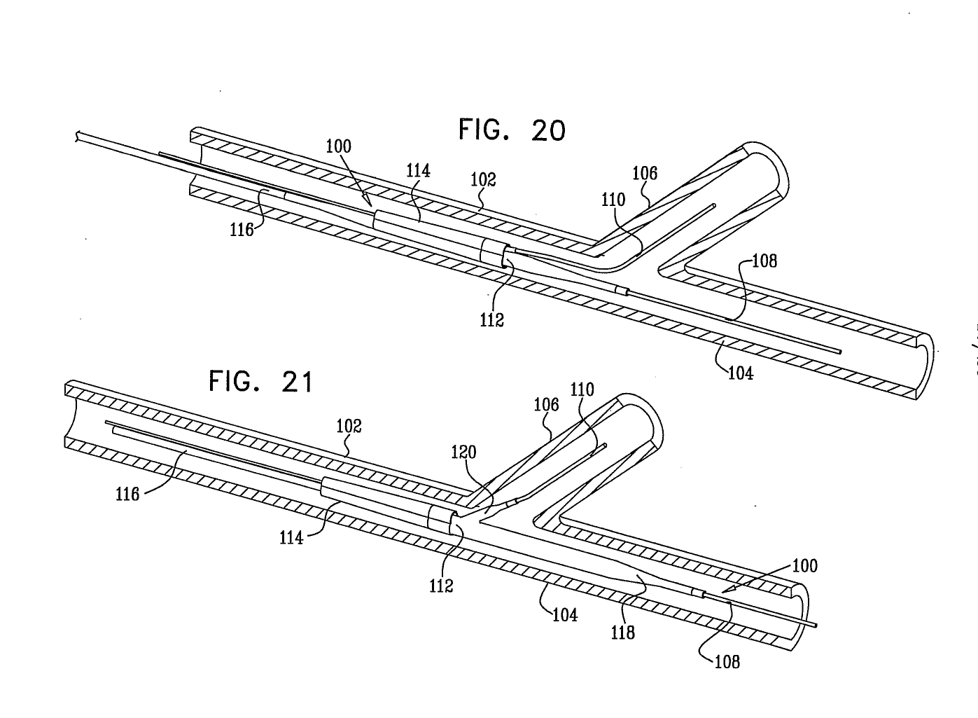

FIGS. 20-22 are schematic, pictorial illustrations showing successive stages in deployment of a system 100 for treatment of a vascular bifurcation, in accordance with an embodiment of the present invention. System 100 comprises a bifurcated balloon 112, which is designed to fit within a bifurcation of a main vessel 102 into branch vessels 104 and 106. (For example, system 100 may be used at the bifurcation of the common carotid artery into internal and external carotid arteries.) Alternatively, branch vessel 104 may simply be a continuation of main vessel 102.

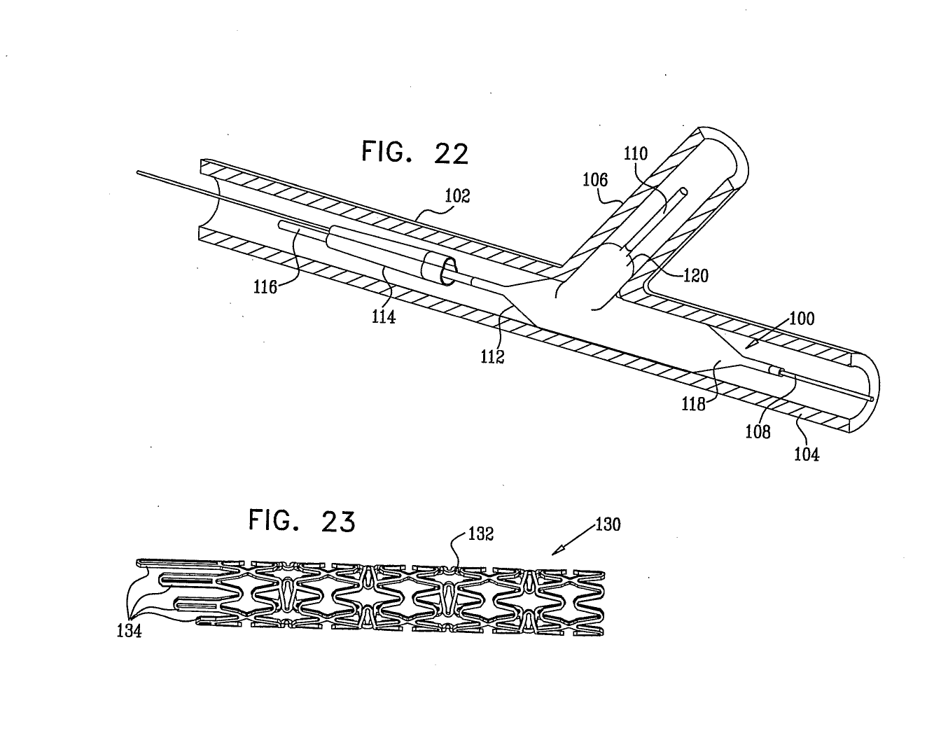

As the first step in deployment of system 100, guide wires 108 and 110 are inserted through vessel 102 into branch vessels 104 and 106, respectively. The operator then deploys system 100 in the area of the bifurcation site by advancing a catheter 116 over the guide wires. During this stage, balloon 112 is contained inside a retainer, in the form of a securing sleeve 114, as shown in FIG. 20. Once the balloon is approximately at the bifurcation site (as indicated by a radio-opaque marker, for example), the operator pulls the sleeve back in the proximal direction, as shown in FIG. 21, thus releasing a side chamber 120 of the balloon. The operator then advances the side chamber further into branch vessel 106 along guide wire 110, while advancing a main chamber 118 of the balloon into branch vessel 104 along guide wire 108. Balloon 112 may then be inflated, as shown in FIG. 22, thus treating both branch vessels 104 and 106 simultaneously. Although chambers 116 and 118 are shown in the figures as two parts of the same balloon, which are inflated and deflated simultaneously, the chambers may alternatively be configured as independently-inflatable parts. Further aspects of treating vascular bifurcations using bifurcated balloons (with or without a stent), which may also be carried out using system 100, are described in the above-mentioned PCT Patent Publication WO 2005/041810 A2.

When treatment is completed, balloon 112 is deflated and is then withdrawn into sleeve 114 for removal from the patient's body. The use of sleeve 114 during insertion and/or removal of the balloon helps to ensure that the bifurcated balloon can be inserted and withdrawn easily from the area of the bifurcation, with minimal risk of damage to the balloon or to the blood vessels during this process.

In an alternative embodiment (not shown in the figures), a spiral coil may be used as the retainer in place of sleeve 114. The spiral coil can be made from a superelastic shape-memory wire (such as NiTi wire), which can also be coated to serve as a radio-opaque marker. The spiral coil is wound so as to contain balloon 112 in roughly the same manner as sleeve 114. To release the balloon in the bifurcation region, the operator pulls the wire in the proximal direction through a dedicated lumen in the catheter. The force of pulling straightens the wire, thus allowing easy retrieval.

FIG. 23 is a schematic side view of a stent 130 for implantation in a vascular bifurcation, in accordance with an embodiment of the present invention. The stent is shown here in a compact configuration, prior to deployment in a blood vessel. Stent 130 comprises a main section 132, having a suitable structure for expansion by a balloon. Struts 134 at the proximal end of the stent are used for anchoring the stent in the bifurcation. The struts are of gradated lengths to accord with the angle of the bifurcation, as shown in the figures that follow. Alternatively, the struts may all be of the same length.

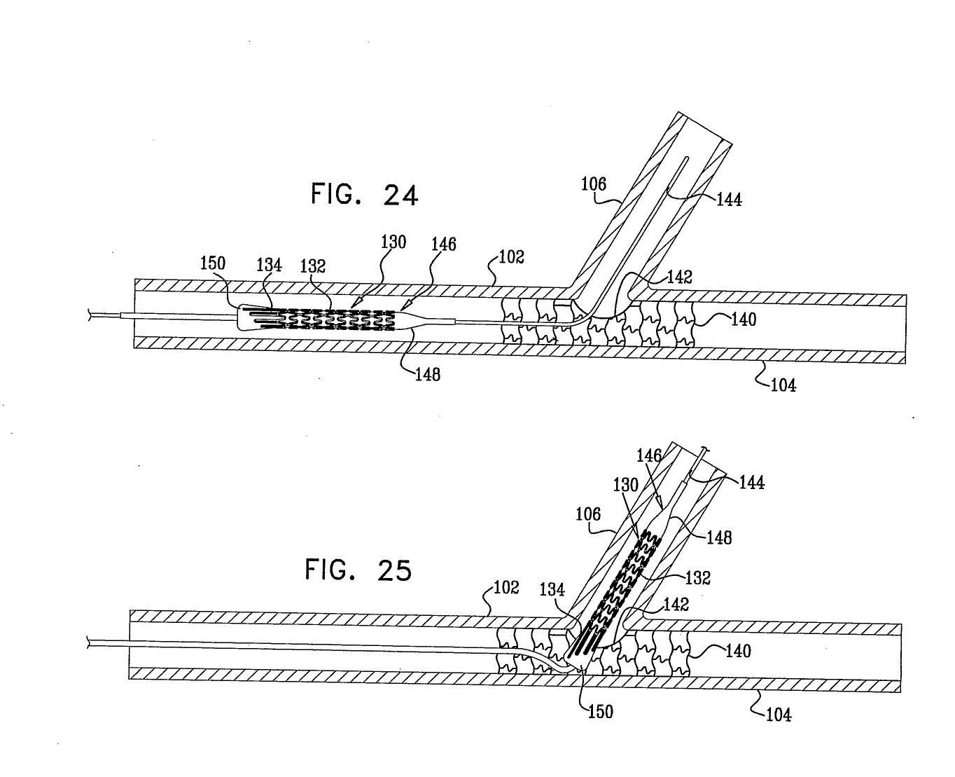

FIGS. 24-27 are schematic, pictorial illustrations showing stages in a procedure for implanting stent 130 in the bifurcation of vessels 104 and 106, in accordance with still another embodiment of the present invention. At the initial stage shown in FIG. 24, a base stent 140 (shown in sectional view in these figures) has already been deployed in vessels 102 and 104. Stent 140 has a lateral opening 142, which is aligned with branch vessel 106. Methods for alignment of this sort are described in the above-mentioned PCT Patent Publication WO 2005/041810 A2. Alternatively, other methods for stent alignment that are known in the art may be used to ensure that lateral opening 142 is in the proper position when stent 140 is expanded into place.

Stent 130 is crimped over a balloon 146, which comprises an inner part 148 and a collar 150, in similar fashion to the two-part balloons described above. Stent 130 and balloon 146 are advanced over a guide wire 144, which is threaded through opening 142 of stent 140 and into branch vessel 106. In this manner, stent 130 is positioned so that inner part 140 protrudes through opening 142, while struts 134 and collar 150 of balloon 146 remain within stent 140, as shown in FIG. 25.

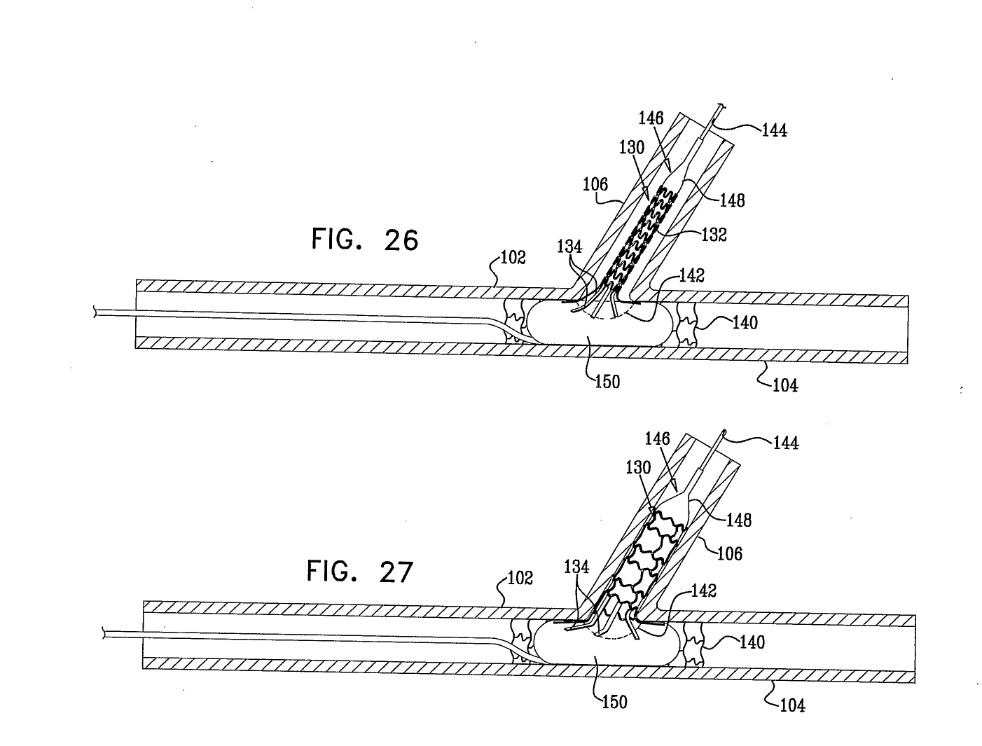

Collar 150 is then inflated, as shown in FIG. 26, thus causing struts 134 to bend outward and engage stent 130 around opening 142. Bending struts 134 in this manner ensures that stent 130 is positioned properly in branch vessel 106. At this point, inner part 148 of balloon 146 is inflated, so that main part 132 of stent 130 expands outward to support vessel 106, as shown in FIG. 27. Bent struts 134 and inflated collar 150 hold stent 130 in the proper location during inflation and anchor stent 130 securely to stent 140, so that the two stents function in the bifurcation as though they were an integral unit. The dual-stent design shown in FIGS. 24-27, however, is easier to manufacture and easier for the practitioner to implant properly than are bifurcated stents that are known in the art.

Alternatively, the operations illustrated in FIGS. 24-27 may be performed in the reverse order. In other words, stent 130 may first be introduced into branch vessel 106, and then deployed and anchored in place (including bending struts 134 outward using collar 150). Thereafter, base stent 140 is inserted and deployed in vessels 102 and 104, thereby engaging struts 134 and anchoring stent 130 in place.

Although the embodiments described above relate mainly to implantation of certain types of stents, the principles of the catheters and balloons used in these embodiments may similarly be applied to stents of other types, as well as to balloon-based vascular treatments that do not involve deployment of a stent. It will thus be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

1. A method for treatment of a vascular bifurcation, where a second blood vessel branches from a first blood vessel, the first and second blood vessels having characteristic first and second diameters such that the first diameter is greater than the second diameter, the method comprising:
introducing a balloon in the first blood vessel into proximity to the vascular bifurcation, the balloon comprising an inner part and a collar proximal to the inner part;
inflating the collar so that the collar expands to an expanded diameter greater than the second diameter but less than an outer diameter of an ostial funnel of the second blood vessel;
advancing the balloon into the second blood vessel so that the inflated collar lodges within the ostial funnel; and
while the inflated collar is lodged within the ostial funnel, inflating the inner part of the balloon so as to treat the second blood vessel.

2. The method according to claim 1, and comprising fitting a stent over the inner part of the balloon, wherein introducing the balloon comprises deploying the stent within the second blood vessel, and wherein inflating the inner part of the balloon causes the stent to expand so that the stent is implanted within the second blood vessel.

3. The method according to claim 2, wherein the stent comprises proximal struts, and wherein inflating the collar causes the proximal struts to expand to a size greater than the second diameter so as to engage the ostial funnel.

4. The method according to claim 3, wherein the expanded struts do not extend into the first blood vessel.

5. The method according to claim 1, wherein introducing the balloon comprises advancing the balloon through the blood vessel using a catheter having a shaft, and inflating the balloon via the shaft, wherein the balloon is formed by fastening the inner part of the balloon around the shaft so as to seal the inner part to the catheter, and fastening the collar around the inner part so as to seal the collar to the inner part.

6. The method according to claim 1, wherein inflating the collar comprises filling the collar with a fluid via a dual-channel manifold, which is also coupled to fill the inner part of the balloon, wherein the manifold is operative to limit a quantity of the fluid with which the collar is filled to a predetermined volume.

7. A method for treatment of a vascular bifurcation, where a second blood vessel branches from a first blood vessel the first and second blood vessels having characteristic first and second diameters such that the first diameter is greater than the second diameter, the method comprising:
fitting a stent over a balloon, which comprises an inner part and a collar proximal to the inner part, the stent comprising proximal struts that extend over the collar;
introducing the balloon, with the stent fitted over the balloon, in the first blood vessel into proximity to the vascular bifurcation;
inflating the collar so as to cause the struts to bend outward to an expanded diameter greater than the second diameter;
advancing the inner part of the balloon, with the stent fitted over the balloon, into the second blood vessel so that the bent struts engage an ostium of the second blood vessel; and
while the bent struts engage the ostium, inflating the inner part of the balloon so as to expand and implant the stent within the second blood vessel.

8. The method according to claim 7, and comprising deflating the collar before the bent struts engage the ostium.

9. The method according to claim 7, wherein introducing the balloon comprises advancing the balloon through the blood vessel using a catheter having a shaft, and inflating the balloon via the shaft, wherein the balloon is formed by fastening the inner part of the balloon around the catheter so as to seal the inner part to the catheter, and fastening the collar around the inner part so as to seal the collar to the inner part.

10. The method according to claim 7, wherein inflating the collar comprises filling the collar with a fluid via a dual-channel manifold, which is also coupled to fill the inner part of the balloon, wherein the manifold is operative to limit a quantity of the fluid with which the collar is filled to a predetermined volume.

11. A method for treatment of a vascular bifurcation, where a second blood vessel branches from a first blood vessel, the first and second blood vessels having characteristic first and second diameters such that the first diameter is greater than the second diameter, the method comprising:
fixing a stent to a distal end of a delivery catheter having an axis, the delivery catheter comprising resilient struts protruding radially outward from the axis at a location proximal to the stent to an expanded diameter greater than the second diameter;
advancing the delivery catheter, with the stent fixed to the distal end thereof, into the first blood vessel through a guiding catheter, thus causing the struts to fold within the guiding catheter in a direction parallel to the axis while the delivery catheter is advanced through the guiding catheter;
introducing the distal end of the delivery catheter, with the stent fixed thereto, distally out of the guiding catheter into proximity to the vascular bifurcation, thus permitting the struts to protrude radially outward from the axis;
advancing the distal end of the delivery catheter, with the stent fitted thereto, into the second blood vessel so that the protruding struts engage an ostium of the second blood vessel; and
while the protruding struts engage the ostium, expanding and implanting the stent within the second blood vessel.

12. The method according to claim 11, wherein fixing the stent comprises fitting the stent over a balloon at the distal end of the delivery catheter, and wherein expanding the stent comprises inflating the balloon.

13. The method according to claim 11, and comprising, after implanting the stent, withdrawing the delivery catheter from the first blood vessel through the guiding catheter, thus causing the struts to fold within the guiding catheter while the delivery catheter is withdrawn.

14. A method for treatment of a vascular bifurcation, where a second blood vessel branches from a first blood vessel, the first and second blood vessels having characteristic first and second diameters such that the first diameter is greater than the second diameter, the method comprising:
fixing a self-expanding stent to a distal end of a catheter having an inflatable collar;
deploying the distal end of the catheter, while holding the stent in a compact state, in the first blood vessel in proximity to the vascular bifurcation;
inflating the collar while the distal end of the catheter is in proximity to the vascular bifurcation so that the collar expands to an expanded diameter greater than the second diameter;
advancing the catheter into the second blood vessel so that the inflated collar engages an ostium of the second blood vessel; and
while the inflated collar engages the ostium, releasing the stent so that the stent expands, thus implanting the stent within the second blood vessel.

15. The method according to claim 14, wherein fixing the self-expanding stent comprises fitting a sleeve over the self-expanding stent so as to hold the stent in the compact state, and wherein releasing the stent comprises withdrawing the sleeve from the second blood vessel while the stent remains within the second blood vessel.

16. The method according to claim 15, wherein withdrawing the sleeve comprises pulling the sleeve through a hub of the collar while the inflated collar engages the ostium.

17. The method according to claim 14, wherein inflating the collar comprises inflating the collar to an expanded diameter greater than the second diameter but less than an outer diameter of an ostial funnel of the second blood vessel, and wherein advancing the catheter comprises advancing the collar into the second blood vessel so that the inflated collar lodges within the ostial funnel.

18. A method for treatment of a vascular bifurcation between first and second blood vessels, the method comprising:
using a catheter, introducing first and second balloon chambers into the first and second blood vessels, respectively, in an area of the bifurcation;
treating the bifurcation by selectively inflating the first and second balloon chambers with a fluid via the catheter using a dual-channel manifold coupled to the catheter; and
configuring the manifold so as to automatically limit a quantity of the fluid with which at least one of the first and second balloon chambers is filled to a predetermined volume.

19. The method according to claim 18, wherein selectively inflating the first and second balloon chambers comprises inflating the first balloon chamber to the predetermined volume, and thereafter inflating the second balloon chamber.

20. The method according to claim 19, and comprising deflating the first and second balloon chambers simultaneously.

21. The method according to claim 18, wherein the first balloon chamber is configured as a collar surrounding a part of the second balloon chamber, and wherein introducing the first and second balloon chambers comprises inserting the second balloon chamber into the second blood vessel, while the part of the balloon surrounded by the collar remains in the first blood vessel, and wherein treating the bifurcation comprises inflating the collar and bringing the inflated collar into engagement with an ostium of the second blood vessel before inflating the second balloon chamber.

22. A method for producing a balloon, comprising:
fastening an inner part of the balloon around the catheter so as to seal the inner part to the catheter; and
forming an inflatable collar around the inner part by fastening a first end of the collar around the inner part so as to seal the collar to the inner part, and fastening a second end of the collar around the catheter so as to seal the collar to the catheter.

23. The method according to claim 22, and comprising providing inflation ports in the catheter through which the inner part and the collar can be independently inflated.

24. The method according to claim 22, wherein fastening the first end of the collar comprises folding the first end inward toward the catheter, and sealing the folded first end to a neck of the inner part of the balloon.

25. A method for treatment of a vascular bifurcation between first and second blood vessels, the method comprising:
fixing first and second balloon chambers to a distal end of a catheter;
fitting a retainer over the first and second balloon chambers;
advancing the distal end of the catheter, while the first and second balloon chambers are contained within the retainer, into a vicinity of the bifurcation;
releasing the first and second balloon chambers from the retainer in the vicinity of the bifurcation; and
after releasing the first and second balloon chambers, deploying and inflating the first and second balloon chambers in the first and second blood vessels, respectively, in order to treat the vascular bifurcation.

26. The method according to claim 25, wherein the retainer comprises a sleeve.

27. The method according to claim 25, wherein the retainer comprises a wire, which is wound around the first and second balloon chambers.

28. The method according to claim 25, wherein releasing the first and second balloon chambers comprises shifting the retainer in a proximal direction relative to the first and second balloon chambers.

29. A method for treatment of a vascular bifurcation where a second blood vessel branches from a first blood vessel, the method comprising:
implanting in the first blood vessel a first stent having a lateral opening, such that the lateral opening is aligned with the second blood vessel;
fitting a second stent over a balloon, which comprises an inner part and a collar proximal to the inner part, the stent comprising proximal struts that extend over the collar;
positioning the balloon, with the second stent fitted over the balloon, in the lateral opening such that the inner part of the balloon protrudes through the lateral opening into the second blood vessel;
inflating the collar within the first blood vessel so as to cause the struts to bend outward and engage the first stent in proximity to the lateral opening, whereby the second stent is positioned at a desired location in the second blood vessel; and
while the bent struts engage the first stent, inflating the inner part of the balloon so as to expand and implant the second stent within the second blood vessel.

30. The method according to claim 29, wherein positioning the balloon comprises inserting a guide wire through the first blood vessel into the second blood vessel via the lateral opening in the first stent, and advancing the balloon over the guide wire.

31. The method according to claim 29, wherein the proximal struts have differing respective lengths, which are chosen responsively to a branching angle of the bifurcation.

32. Apparatus for treatment of a vascular bifurcation, where a second blood vessel branches from a first blood vessel, the first and second blood vessels having characteristic first and second diameters such that the first diameter is greater than the second diameter, the apparatus comprising:
a balloon, comprising an inner part and a collar proximal to the inner part, wherein the collar is coupled to be inflated to an expanded diameter greater than the second diameter but less than an outer diameter of an ostial funnel of the second blood vessel; and
a catheter, which has a distal end to which the balloon is fixed, and which is configured for insertion through the first blood vessel into proximity with the vascular bifurcation and is operable to inflate the collar and to advance the balloon into the second blood vessel so that the inflated collar lodges within the ostial funnel, and to inflate the inner part of the balloon while the inflated collar is lodged within the ostial funnel so as to treat the second blood vessel.

33. The apparatus according to claim 32, and comprising a stent, which is fitted over the inner part of the balloon, wherein inflating the inner part of the balloon causes the stent to expand so that the stent is implanted within the second blood vessel.

34. The apparatus according to claim 33, wherein the stent comprises proximal struts, and wherein inflating the collar causes the proximal struts to expand to a size greater than the second diameter so as to engage the ostial funnel.

35. The apparatus according to claim 34, wherein the expanded struts do not extend into the first blood vessel.

36. The apparatus according to claim 32, wherein the balloon is formed by fastening the inner part of the balloon around the catheter so as to seal the inner part to the catheter, and fastening the collar around the inner part so as to seal the collar to the inner part.

37. The apparatus according to claim 32, wherein the catheter has first and second inflation lumens, which are respectively coupled to the inner part of the balloon and the collar, and comprising a dual-channel manifold, which is coupled via the inflation lumens to fill the collar and the inner part of the balloon with a fluid, wherein the manifold is operative to limit a quantity of the fluid with which the collar is filled to a predetermined volume.

38. Apparatus for treatment of a vascular bifurcation, where a second blood vessel branches from a first blood vessel, the first and second blood vessels having characteristic first and second diameters such that the first diameter is greater than the second diameter, the apparatus comprising:
a balloon, which is fixed to the distal end of the catheter and comprises an inner part and a collar proximal to the inner part;
a stent, which is fitted over the inner part of the balloon and comprises proximal struts that extend over the collar; and
a catheter, which has a distal end to which the balloon is fixed, and which is configured for insertion through the first blood vessel into proximity with the vascular bifurcation and is operable to deploy the balloon, with the stent fitted over the balloon, in the first blood vessel in proximity to the vascular bifurcation, to inflate the collar so as to cause the struts to bend outward to an expanded diameter greater than the second diameter, then to advance the inner part of the balloon, with the stent fitted over the balloon, into the second blood vessel so that the bent struts engage an ostium of the second blood vessel, and to inflate the inner part of the balloon while the bent struts engage the ostium so as to expand and implant the stent within the second blood vessel.

39. The apparatus according to claim 38, wherein the catheter is operative to deflate the collar before the bent struts engage the ostium.

40. The apparatus according to claim 38, wherein the balloon is formed by fastening the inner part of the balloon around the catheter so as to seal the inner part to the catheter, and fastening the collar around the inner part so as to seal the collar to the inner part.

41. The apparatus according to claim 38, wherein the catheter has first and second inflation lumens, which are respectively coupled to the inner part of the balloon and the collar, and comprising a dual-channel manifold, which is coupled via the inflation lumens to fill the collar and the inner part of the balloon with a fluid, wherein the manifold is operative to limit a quantity of the fluid with which the collar is filled to a predetermined volume.

42. Apparatus for treatment of a vascular bifurcation, where a second blood vessel branches from a first blood vessel, the first and second blood vessels having characteristic first and second diameters such that the first diameter is greater than the second diameter, the apparatus comprising:
a stent;
a guiding catheter, which is configured to be introduced into the first blood vessel in proximity to the vascular bifurcation; and
a delivery catheter, which has an axis and a distal end to which the stent is fixed, and which is configured to pass through the guiding catheter into the first blood vessel, and which comprises resilient struts at a location proximal to the stent,
wherein passing the delivery catheter through the guide catheter causes the struts to fold in a direction parallel to the axis, and wherein upon emergence of the distal end of the delivery catheter distally out of the guiding catheter in proximity to the vascular bifurcation, the resilient struts protrude radially outward from the axis and engage an ostium of the second blood vessel while the stent is inserted into the second blood vessel.

43. The apparatus according to claim 42, and comprising a balloon fixed to the distal end of the delivery catheter, where the stent is fitted over the balloon, and wherein the balloon is inflatable so as to expand and implant the stent in the second blood vessel.

44. The apparatus according to claim 42, wherein the delivery catheter is adapted to be withdrawn from the first blood vessel through the guiding catheter, thus causing the struts to fold within the guiding catheter while the delivery catheter is withdrawn.

45. The apparatus according to claim 42, and comprising a sleeve fitted around the delivery catheter so as to hold the resilient struts parallel to the axis while passing the delivery catheter through the guide catheter.

46. Apparatus for treatment of a vascular bifurcation, where a second blood vessel branches from a first blood vessel, the first and second blood vessels having characteristic first and second diameters such that the first diameter is greater than the second diameter, the apparatus comprising:
a self-expanding stent; and
a catheter, having a distal end to which the stent is fixed in a compact state and which is configured for insertion through the first blood vessel into proximity with the vascular bifurcation so that the stent is inserted into the second blood vessel, the catheter comprising an inflatable collar proximal to the distal end,
wherein the catheter is operable to inflate the collar while the distal end of the catheter is in proximity to the vascular bifurcation so that the collar expands to an expanded diameter greater than the second diameter and engages an ostium of the second blood vessel while the stent is released within the second blood vessel so that the stent expands, thus implanting the stent within the second blood vessel.

47. The apparatus according to claim 46, and comprising a sleeve, which is fitted over the self-expanding stent so as to hold the stent in the compact state, wherein the sleeve is withdrawn from the second blood vessel in order to release the stent while the stent remains within the second blood vessel.

48. The apparatus according to claim 47, wherein the catheter is operable to withdraw the sleeve by pulling the sleeve through a hub of the collar while the inflated collar engages the ostium.

49. The apparatus according to claim 46, wherein the collar is inflatable to an expanded diameter greater than the second diameter but less than an outer diameter of an ostial funnel of the second blood vessel, so that the inflated collar lodges within the ostial funnel.

50. Apparatus for vascular treatment, the apparatus comprising:
first and second balloon chambers;
a catheter, having distal and proximal ends and containing first and second lumens coupled respectively to the first and second balloon chambers at the distal end of the catheter; and
a manifold comprising first and second fluid channels coupled respectively to the first and second lumens at the proximal end of the catheter and operative to selectively inflate the first and second balloon chambers with a fluid via the catheter while automatically limiting a quantity of the fluid with which at least one of the first and second balloon chambers is filled to a predetermined volume.

51. The apparatus according to claim 50, wherein the manifold comprises a switch for selecting one of the first and second fluid channels for inflation.

52. The apparatus according to claim 51, wherein the manifold is operative to deflate the first and second balloon chambers simultaneously irrespective of the switch.

53. The apparatus according to claim 50, wherein the manifold comprises a valve, for shutting off a flow of the fluid to the at least one of the first and second balloon chambers, and a flow meter, which is operative to measure the flow through the at least one of the first and second balloon chambers and to actuate the valve when the predetermined volume has passed.

54. The apparatus according to claim 50, wherein the first balloon chamber is configured as a collar surrounding a part of the second balloon chamber.

55. A medical device, comprising:
a catheter having a distal end; and
a dual-chamber balloon fixed to the distal end of the catheter, the balloon comprising:
an inner part, which is fastened around the distal end of the catheter so as to seal the inner part to the catheter; and
an inflatable collar, which is formed around the inner part by fastening a first end of the collar around the inner part so as to seal the collar to the inner part, and fastening a second end of the collar around the catheter so as to seal the collar to the catheter.

56. The device according to claim 55, wherein the catheter contains a lumen and inflation ports through which the inner part and the collar can be independently inflated.

57. The device according to claim 55, wherein the first end of the collar is folded inward toward the catheter and is sealed to a neck of the inner part of the balloon.

58. Apparatus for treatment of a vascular bifurcation between first and second blood vessels, the apparatus comprising:
a catheter having a distal end;
first and second balloon chambers, which are fixed to the distal end of a catheter; and
a retainer, which fits over and contains the first and second balloon chambers while the distal end of the catheter is advanced into a vicinity of the bifurcation, and which is operable to release the first and second balloon chambers in the vicinity of the bifurcation, so that the first and second balloon chambers can be deployed and inflated in the first and second blood vessels, respectively, in order to treat the vascular bifurcation.

59. The apparatus according to claim 58, wherein the retainer comprises a sleeve.

60. The apparatus according to claim 58, wherein the retainer comprises a wire, which is wound around the first and second balloon chambers.

61. The apparatus according to claim 58, wherein the retainer is shifted in a proximal direction relative to the first and second balloon chambers in order to release the first and second balloon chambers.

62. Apparatus for treatment of a vascular bifurcation where a second blood vessel branches from a first blood vessel, the apparatus comprising:
a first stent, which has a lateral opening and is configured to be implanted in the first blood vessel such that the lateral opening is aligned with the second blood vessel;
a balloon, which comprises an inner part and a collar proximal to the inner part;
a second stent, which comprises proximal struts and is fitted over the balloon so that the struts extend over the collar; and
a catheter, having a distal end to which the balloon is fixed, and which is operative to position the balloon, with the second stent fitted over the balloon, in the lateral opening such that the inner part of the balloon protrudes through the lateral opening into the second blood vessel, and to inflate the collar within the first blood vessel so as to cause the struts to bend outward and engage the first stent in proximity to the lateral opening, and to inflate the inner part of the balloon while the bent struts engage the first stent so as to expand and implant the second stent within the second blood vessel.

63. The apparatus according to claim 62, and comprising a guide wire, which is adapted to be inserted through the first blood vessel into the second blood vessel via the lateral opening in the first stent, wherein the catheter is configured to be advanced over the guide wire.

64. The apparatus according to claim 62, wherein the proximal struts have differing respective lengths, which are chosen responsively to a branching angle of the bifurcation.

65. A method using first and second stents for treatment of a vascular bifurcation where a first blood vessel branches from a second blood vessel, the first stent having a lateral opening, the method comprising:
fitting the second stent over a balloon, which comprises an inner part and a collar proximal to the inner part, the stent comprising proximal struts that extend over the collar;
positioning the balloon, with the second stent fitted over the balloon, in the vascular bifurcation so that the inner part of the balloon protrudes into the second blood vessel while the collar is in the first blood vessel;
after positioning the balloon, inflating the collar within the first blood vessel so as to cause the struts to bend outward and engage a wall of the first blood vessel in proximity to the bifurcation, whereby the second stent is positioned at a desired location in the second blood vessel; and
while the bent struts engage the wall of the first blood vessel, inflating the inner part of the balloon so as to expand and implant the second stent within the second blood vessel; and
after implanting the second stent within the second blood vessel, implanting the first stent in the first blood vessel, such that the lateral opening is aligned with the second blood vessel and engages the bent struts.

66. Apparatus for treatment of a vascular bifurcation where a second blood vessel branches from a first blood vessel, the apparatus comprising:
a first stent, which has a lateral opening and is configured to be implanted in the first blood vessel such that the lateral opening is aligned with the second blood vessel;
a balloon, which comprises an inner part and a collar proximal to the inner part;
a second stent, which comprises proximal struts and is fitted over the balloon so that the struts extend over the collar; and
a catheter, having a distal end to which the balloon is fixed, and which is operative to position the balloon, with the second stent fitted over the balloon, in the vascular bifurcation so that the inner part of the balloon protrudes into the second blood vessel while the collar is in the first blood vessel, and to inflate the collar within the first blood vessel so as to cause the struts to bend outward and engage a wall of the first blood vessel in proximity to the bifurcation, whereby the second stent is positioned at a desired location in the second blood vessel, and to inflate the inner part of the balloon while the bent struts engage the wall of the first blood vessel so as to expand and implant the second stent within the second blood vessel,
wherein the first stent is implanted in the first blood vessel after implanting the second stent within the second blood vessel, such that the lateral opening is aligned with the second blood vessel and engages the bent struts.
